(19) United States
(12) Patent Application Publication
(10) Pub. No.: US 2026/0151435 A1
(43) Pub. Date: Jun. 4, 2026

(54) METHOD FOR DRYING FECAL MICROBIOTA MULTILAYER DRIPPING PILLS

(71) Applicant: SHENZHEN WANHE PHARMACEUTICAL CO., LTD., Shenzhen (CN)

(72) Inventors: Zhongsun WANG, Shenzhen (CN); Yang JIN, Shenzhen (CN); Baisheng KE, Shenzhen (CN); Weijia HUANG, Shenzhen (CN); Jun MA, Shenzhen (CN)

(21) Appl. No.: 19/460,874

(22) Filed: Jan. 27, 2026

Related U.S. Application Data
(63) Continuation of application No. PCT/CN2024/123692, filed on Oct. 9, 2024.

(30) Foreign Application Priority Data
Sep. 29, 2024 (CN) 202411369203.8

(51) Int. Cl. A61K 35/37; A61K 9/50; A61K 35/741

(52) U.S. Cl. CPC A61K 35/37; A61K 9/501; A61K 9/5015; A61K 9/5057; A61K 9/5073; A61K 9/5089; A61K 35/741

(57) ABSTRACT

A method for drying fecal microbiota multilayer dripping pills is disclosed. The drying method includes: performing centrifugal deoiling on fecal microbiota multilayer dripping pills by using a deoiling agent, and then performing separation treatment to obtain deoiled dripping pills, wherein the deoiling agent is a mixture of fumed silica, soybean fiber, and oat cellulose; performing blast drying on the deoiled dripping pills to obtain pre-dried dripping pills; and performing negative pressure drying on the pre-dried dripping pills to obtain Huanglong dripping pills. In the drying method provided by the present application, centrifugal deoiling, blast drying, and negative pressure drying are sequentially performed, thereby further reducing the water activity of the Huanglong dripping pills. The data of examples show that the obtained Huanglong dripping pills have a water activity aw of 0.08 to 0.14, exhibit excellent tolerability, particularly in vitro simulated gastrointestinal tolerability, and also exhibit excellent stability.

### CROSS-REFERENCE TO RELATED APPLICATIONS

The present application is a continuation application of International Patent Application No. PCT/CN2024/123692, filed on Oct. 9, 2024, which claims priority to Chinese Patent Application No. 202411369203.8, filed with the China National Intellectual Property Administration on Sep. 29, 2024 and entitled “METHOD FOR DRYING FECAL MICROBIOTA MULTILAYER DRIPPING PILLS”, each of which is incorporated herein by reference in its entirety.

### TECHNICAL FIELD

The present application relates to the technical field of pharmaceutical preparation, in particular to a method for drying fecal microbiota multilayer dripping pills.

### BACKGROUND

“Fecal microbiota transplantation” (FMT) refers to the transplantation of functional microbiota from healthy human feces into the intestinal tract of a patient to reconstruct new gut microbiota, thereby treating intestinal and extraintestinal diseases. It is used to treat intestinal diseases such as Clostridium difficile infection, refractory diarrhea, constipation, gastroenteritis, Crohn's disease and ulcerative gastroenteritis, extraintestinal diseases such as obesity, asthma, autism spectrum disorder, depression, diabetes mellitus and cancers, and other refractory conditions.

Fecal microbiota transplantation administration routes are divided into three approaches: upper gastrointestinal tract, middle gastrointestinal tract, and lower gastrointestinal tract. The upper gastrointestinal tract approach mainly refers to oral administration of fecal microbiota capsules, which has the advantages of non-invasiveness and high patient acceptance. The middle gastrointestinal tract approach mainly includes nasogastric tube, nasojejunal tube, and nasoduodenal tube administration, and is particularly suitable for patients with colonic inflammation. However, intubation can cause patient discomfort, and this approach does not allow for evaluation of intestinal mucosa in patients and collection of tissue samples. The lower gastrointestinal tract approach mainly includes colonoscopy and retention enema. Compared to intubation, this approach reduces patient discomfort. Furthermore, due to the need for preoperative bowel cleansing, localization of colonic condition can be achieved, and beneficial microbiota is delivered to targeted sites. However, the disadvantage is delivery only at distal colon. The above three approaches have comparable therapeutic effects, oral administration of fecal microbiota capsules is the most patient-preferred, and therefore it has the broadest application prospects.

Using fecal microbiota lyophilized powder as a raw material, a novel dripping pill preparation technology is used to prepare seamless dripping pills, and the seamless dripping pills are then dried to obtain Huanglong dripping pills. The in vitro disintegration experiment results show that the Huanglong dripping pills remain intact after 2 hours in an acidic environment at pH 1.2, and still have good integrity after 4 h in an environment at pH 6.8, and the storage conditions are simple. Water activity is critical for the efficacy and storage of Huanglong dripping pills. If the water activity of Huanglong dripping pills is high, the storage of the Huanglong dripping pills is inconvenient, and the efficacy is affected. Drying seamless dripping pills is a key step influencing the water activity of Huanglong dripping pills. The research focus of the current preparation of Huanglong dripping pills lies in simultaneously ensuring the viability of the lyophilized microbiota powder and the low water activity in Huanglong dripping pills.

The Chinese patent (publication No. CN 115671067 A) discloses the use of Huanglong dripping pills in the treatment and prevention of irritable bowel syndrome, in which multilayer materials are dripped into a cooling liquid to obtain seamless dripping pills having a concentric structure, and the obtained seamless dripping pills having a concentric structure are then subjected to blast drying at 15° C. to 20° C. for 10 to 12 hours to remove outer-layer moisture to obtain the dripping pills. The Huanglong dripping pills obtained by the above drying method have a high water activity (aw), being 0.15 to 0.20.

### SUMMARY

In view of this, an objective of the present application is to provide a method for drying fecal microbiota multilayer dripping pills. The Huanglong dripping pills obtained by the drying method provided by the present application have a low water activity.

In order to achieve the above objective of the present application, the present application provides the following technical solutions.

The present application provides a method for drying fecal microbiota multilayer dripping pills, including the steps of:
providing fecal microbiota multilayer dripping pills;
performing centrifugal deoiling on the fecal microbiota multilayer dripping pills by using a deoiling agent, and then performing separation treatment to obtain deoiled dripping pills, where the deoiling agent is a mixture of fumed silica, soybean fiber, and oat cellulose;
performing blast drying on the deoiled dripping pills to obtain pre-dried Huanglong dripping pills; and
performing negative pressure drying on the pre-dried Huanglong dripping pills to obtain Huanglong dripping pills.

Preferably, the mass ratio of the fumed silica to the soybean fiber to the oat cellulose is 1:1 to 2:1 to 2.

Preferably, the mass ratio of the fecal microbiota multilayer dripping pills to the deoiling agent is 1:1 to 3.

Preferably, in the centrifugal deoiling, the rotating speed is 500 to 1200 revolutions per minute (rpm), and the time is 10 to 15 minutes (min).

Preferably, the time of the separation treatment is 3 to 8 min;
the separation treatment is performed in a rotary drying drum, and the rotating speed of the rotary drying drum is 5 to 10 rpm.

Preferably, in the blast drying, the rotating speed is 4 to 10 rpm, and the time is 5.5 to 7.5 hours (h); the blast gas of the blast drying is sterile air;
the blast drying is performed in a rotary drying drum, the drum wall of the rotary drying drum is provided with pores having a pore size of 0.5 to 1.5 mm, and the rotary drying drum is lined with a pure cotton oil-absorbing cloth;
during the first 60 min of the blast drying, the pure cotton oil-absorbing cloth is replaced every 30 min.

Preferably, the pre-dried Huanglong dripping pills have a water activity of 0.15 to 0.2.

Preferably, in the negative pressure drying, the pressure is 0 to 0.02 MPa, the temperature is 25° C. to 28° C., the humidity is 3 to 15%, and the time is 60 to 180 min.

Preferably, the negative pressure drying is performed in a rotary drying drum, and the rotating speed of the rotary drying drum is 4 to 10 rpm;
the rotary drying drum is filled with a desiccant, the desiccant is a mixture of a color changing silica gel and anhydrous calcium chloride, and the mass ratio of the color changing silica gel to the anhydrous calcium chloride is 1:1 to 3.

Preferably, the Huanglong dripping pills have a water activity of 0.08 to 0.14.

The present application provides a method for drying fecal microbiota multilayer dripping pills, including the steps of: providing fecal microbiota multilayer dripping pills; performing centrifugal deoiling on the fecal microbiota multilayer dripping pills by using a deoiling agent, and then performing separation treatment to obtain deoiled dripping pills, where the deoiling agent is a mixture of fumed silica, soybean fiber, and oat cellulose; performing blast drying on the deoiled dripping pills to obtain pre-dried Huanglong dripping pills; and performing negative pressure drying on the pre-dried Huanglong dripping pills to obtain Huanglong dripping pills.

In the drying method provided by the present application, centrifugal deoiling, blast drying, and negative pressure drying are sequentially performed, thereby further reducing the water activity of the Huanglong dripping pills. The data of examples show that the obtained Huanglong dripping pills have a water activity (aw) of 0.08 to 0.14, exhibit excellent tolerability, particularly in vitro simulated gastrointestinal tolerability, and also exhibit excellent stability.

Further, the drying method provided by the present application takes a short time and has a high working effect.

### DETAILED DESCRIPTION OF THE EMBODIMENTS

The present application provides a method for drying fecal microbiota multilayer dripping pills, including the steps of:
providing fecal microbiota multilayer dripping pills;
performing centrifugal deoiling on the fecal microbiota multilayer dripping pills by using a deoiling agent, and then performing separation treatment to obtain deoiled dripping pills, where the deoiling agent is a mixture of fumed silica, soybean fiber, and oat cellulose;
performing blast drying on the deoiled dripping pills to obtain pre-dried Huanglong dripping pills;
performing negative pressure drying on the pre-dried Huanglong dripping pills to obtain Huanglong dripping pills.

In the present application, unless otherwise specified, the raw materials used in the present application are preferably commercially available products.

The present application provides fecal microbiota multilayer dripping pills.

In the present application, the fecal microbiota multilayer dripping pills refer to undried Huanglong dripping pills, where the multilayer structure of the undried Huanglong dripping pills is already formed but not yet dried. In the present application, there are no specific limitations on the method for preparing the fecal microbiota multilayer dripping pills, so long as the operation is well known to those skilled in the art.

In the present application, after providing fecal microbiota multilayer dripping pills, centrifugal deoiling is performed on the fecal microbiota multilayer dripping pills by using a deoiling agent, and separation treatment is then performed to obtain deoiled dripping pills, where the deoiling agent is a mixture of fumed silica, soybean fiber, and oat cellulose.

In the present application, the mass ratio of the fumed silica to the soybean fiber to the oat cellulose is preferably 1:1 to 2:1 to 2, further preferably 1:1:1. In the present application, a method for preparing the deoiling agent preferably includes the steps of: mixing fumed silica, soybean fiber, and oat cellulose, and performing vacuum drying and cooling to obtain the deoiling agent, where in the vacuum drying, the temperature is preferably 40° C. to 60° C., further preferably 50° C., the time is preferably 4 to 6 h, further preferably 5 h, and the vacuum drying is preferably performed in a vacuum drying oven. In the present application, the mass ratio of the fecal microbiota multilayer dripping pills to the deoiling agent is preferably 1:1 to 3, specifically preferably 1:1.

In the present application, in the centrifugal deoiling, the rotating speed is preferably 500 to 1200 rpm, further preferably 800 to 1000 rpm, and the time is preferably 10 to 15 min, further preferably 11 to 14 min, more preferably 12 to 13 min. In the present application, the centrifugal deoiling preferably includes: loading the deoiling agent and the fecal microbiota multilayer dripping pills into a sterile bag, and then placing the sterile bag in a centrifuge for centrifugal deoiling.

In the present application, the time of the separation treatment is preferably 3 to 8 min. In the present application, the separation treatment is preferably performed in a rotary drying drum, and the drum wall of the rotary drying drum is preferably provided with pores preferably having a pore size of 0.5 to 1.5 mm; the rotating speed of the rotary drying drum is preferably 5 to 10 rpm. In the present application, the drum of the rotary drying drum is preferably subjected to pretreatment before use, the pretreatment preferably includes sequentially performing boiling in boiling water and blow drying, the time of the boiling in boiling water is preferably 30 to 45 min, the gas of the blow drying is preferably sterile air, and the pressure of the sterile air is preferably 0.2 to 0.3 MPa. In the present application, the separation treatment enables the separation of the deoiling agent from the deoiled dripping pills.

In the present application, after obtaining the deoiled dripping pills, blast drying is performed on the deoiled dripping pills to obtain pre-dried Huanglong dripping pills.

In the present application, in the blast drying, the rotating speed is preferably 4 to 10 rpm, and the time is preferably 5.5 to 7.5 h. In the present application, the blast gas of the blast drying is preferably sterile air.

In the present application, the blast drying is preferably performed in a rotary drying drum, the drum wall of the rotary drying drum is preferably provided with pores preferably having a pore size of 0.5 to 1.5 mm, and the rotary drying drum is preferably lined with a pure cotton oil-absorbing cloth. In the present application, the pure cotton oil-absorbing cloth is preferably subjected to pretreatment before use, the pretreatment includes sequentially performing steam sterilization and drying, in the steam sterilization, the temperature is preferably 121° C. and the time is preferably 20 min, and in the drying, the temperature is preferably 45° C. to 50° C. and the time is preferably 2 to 3 h. In the present application, the drum of the rotary drying drum is preferably subjected to pretreatment before use, the pretreatment preferably includes sequentially performing boiling in boiling water and blow drying, the time of the boiling in boiling water is preferably 30 to 45 min, the gas of the blow drying is preferably sterile air, and the pressure of the sterile air is preferably 0.2 to 0.3 MPa.

In the present application, during the first 60 min of the blast drying, the pure cotton oil-absorbing cloth is preferably replaced every 30 min.

In the present application, the pre-dried Huanglong dripping pills preferably have a water activity of 0.15 to 0.2.

In the present application, after obtaining the pre-dried Huanglong dripping pills, negative pressure drying is performed on the pre-dried Huanglong dripping pills to obtain Huanglong dripping pills.

In the present application, in the negative pressure drying, the pressure is preferably 0 to 0.02 MPa, the temperature is preferably 25° C. to 28° C., the humidity is preferably 3 to 15%, and the time is preferably 60 to 180 min.

In the present application, the negative pressure drying is preferably performed in a rotary drying drum, and the rotating speed of the rotary drying drum is preferably 4 to 10 rpm.

In the present application, the rotary drying drum is preferably filled with a desiccant, the desiccant is preferably a mixture of a color changing silica gel and anhydrous calcium chloride, and the mass ratio of the color changing silica gel to the anhydrous calcium chloride is preferably 1:1 to 3. In the present application, the color changing silica gel and the anhydrous calcium chloride preferably have particle sizes of 5 to 8 mm, independently. In the present application, a method for preparing the desiccant preferably includes the steps of: mixing a color changing silica gel and anhydrous calcium chloride, and performing drying to obtain the desiccant, where in the drying, the temperature is preferably 150° C. to 170° C. and the time is preferably 6 to 8 h.

After the negative pressure drying, the present application preferably further includes sieving the obtained material. In the present application, the sieving is preferably performed in an anaerobic glove box.

In the present application, the Huanglong dripping pills preferably have a water activity of 0.08 to 0.14; the Huanglong dripping pills preferably have a particle size of 1.8 to 2.5 mm.

The method for drying fecal microbiota multilayer dripping pills provided by the present application will be described in detail in conjunction with the examples below, which cannot be construed as limiting the scope of protection of the present application.

### Example 1

First, 200 g of fumed silica, 200 g of soybean fiber, and 200 g of oat cellulose were mixed. The resulting mixture was subjected to vacuum drying at 50° C. for 5 h, then cooled to room temperature, and loaded into a sterile bag.

Then, 600 g of fecal microbiota multilayer dripping pills prepared by a dripping pill device were loaded into the sterile bag, and centrifugal deoiling was performed at 1000 rpm for 12 min in a centrifuge.

Pretreatment was performed on a rotary drying drum, involving: the rotary drying drum was boiled in boiling water for 45 min, and then subjected to blow drying with sterile filtered air at 0.3 MPa.

After the centrifugal deoiling, the materials in the sterile bag were poured into the pretreated rotary drying drum (with a pore size of 0.5 to 1.5 mm), and separated for 5 min at a rotating speed of 6 rpm. The deoiling agent was removed by sieving to obtain deoiled dripping pills.

The deoiled dripping pills were placed in the rotary drying drum (the rotary drying drum was pretreated before use, and the pretreatment was boiling the rotary drying drum in boiling water for 45 min and then performing blow drying with sterile filtered air at 0.3 MPa; and the rotary drying drum was lined with a pure cotton oil-absorbing cloth, which was subjected to steam sterilization at 121° C. for 20 min and drying at 45° C. for 2.5 h before use), and blast drying was performed at 6 rpm for 6.5 h under the condition of blasting sterile air to obtain pre-dried Huanglong dripping pills, where during the first 60 min, the pure cotton oil-absorbing cloth was replaced every 30 min. The water activity aw of the pre-dried Huanglong dripping pills was detected as 0.158.

200 g of color changing silica gel (particle size: 6 mm) and 200 g of anhydrous calcium chloride (particle size: 6 mm) were mixed, dried at 115° C. for 2.5 h, and cooled to room temperature to obtain a desiccant. The desiccant was loaded into the rotary drying drum (the rotary drying drum was pretreated before use, and the pretreatment was boiling the rotary drying drum in boiling water for 45 min and then performing blow drying with sterile filtered air at 0.3 MPa). The rotary drying drum was controlled at a temperature of 28° C., a humidity of 5%, and a rotating speed of 5 rpm. Vacuum was applied to maintain a negative pressure of 0.02 MPa in the rotary drying drum for negative pressure drying for 120 min to obtain Huanglong dripping pills. The water activity aw of the Huanglong dripping pills was detected as 0.092.

The final Huanglong dripping pills were sieved in an anaerobic glove box to obtain Huanglong dripping pills 1 with a particle size of 1.8 to 2.5 mm.

The method for preparing the fecal microbiota multilayer dripping pills included the steps of:

### 1. Outer Layer Matrix

Formulation of outer layer matrix: gelatin (bloom strength 160) 25 wt %, glycerol 1.5 wt %, sucrose 4 wt %, water q.s. to 100 wt %. Preparation of outer layer matrix: gelatin was soaked in an appropriate amount of water (overnight) for dissolving, glycerol and sucrose were added, stirring was performed until dissolved, and water was added to the total amount to obtain the outer layer matrix.

### 2. Middle Layer Matrix

Formulation of middle layer matrix: hardened fat (melting point 39° C. to 43° C., measured 43° C.) 93 wt %, natural vitamin E oil 3 wt %, calcium chloride 4 wt %. Preparation of middle layer matrix: various materials were uniformly mixed, melted at 60° C., and homogenized using a homogenizer at 5000 r/min to obtain the middle layer matrix.

### 3. Inner Layer Core Material

Formulation of inner layer core material: bioactive substances (such as probiotics, gut microbiota, enzymes, and polypeptides, added at an amount of 10 to 30%; in this example, the gut microbiota powder was obtained by impurity removal and lyophilization treatment of feces provided by healthy volunteer B, added at an amount of 20 wt %), natural vitamin E oil 1.8 wt %, dipotassium hydrogen phosphate-potassium dihydrogen phosphate (1:5) 1.2 wt %, vegetable oil (melting point 37° C. to 42° C., measured 38° C.) q.s. to 100% (added at an amount of 60 to 80%, adjusted on the basis of the amount of bioactive substances added). Preparation of inner layer core material: vegetable oil was melted, natural vitamin E oil and micronized dipotassium hydrogen phosphate-potassium dihydrogen phosphate were added, uniform mixing was performed, bioactive substances were then added, homogenization was performed using a homogenizer at 5000 r/min, the temperature was maintained, and stirring was performed to prevent sedimentation for later use.

### 4. Pill Formation

The weight ratio of the outer layer/middle layer/inner layer was 1:3:1.5. Using a three-layer dripping pill device, the inner layer material, the middle layer material, and the outer layer material were separately dripped into a cooling liquid (liquid paraffin) at 18° C. to 20° C. from the inner layer channel, middle layer channel and outer layer channel of a three-channel nozzle (the temperature of the nozzle was maintained at 65° C.) for cooling to form seamless dripping pills having a three-layer concentric structure (the obtained dripping pills had a diameter of 2.5 mm), i.e., the fecal microbiota multilayer dripping pills.

### Comparative Example 1

600 g of fecal microbiota multilayer dripping pills prepared by the dripping pill device were dried at 18° C. for 11 h to obtain Huanglong dripping pills 2, where the fecal microbiota multilayer dripping pills were taken from the fecal microbiota multilayer dripping pills prepared in Example 1 (same batch).

### Test Example 1

Stability experiment of Huanglong dripping pills at 25° C. (0 to 28 days (d))
Subpackaging machine: back sealing granule packaging machine, model DXDK50IIE1, manufactured by ZHUHAI RUICHUANG TECHNOLOGIES DEVELOPMENT CO., LTD.
Aluminum foil bag: material PET12/AL7/PET12/PE80, ZHEJIANG GOLDSTONE PACKAGING CO., LTD.
Oxygen absorber: active substance content 99%, batch number 20210201, NANJING WEINUO ANTISTALING AGENT FACTORY.
Desiccant: active substance content 99%, batch number 20210201, NANJING WEINUO ANTISTALING AGENT FACTORY.
Dripping pill preparation and drying date: Jul. 27, 2021.
Dripping pill storage conditions: (25° C.±2° C./60% RH±5% RH).

The results are as shown in Table 1 and Table 2.

TABLE 1
Water activity experiment results of Huanglong
dripping pills at 25° C. (0 to 28 days)

| 25° C. water activity | day 0 | day 2 | day 4 | day 7 | day 10 | day 14 | day 28 |
|---|---|---|---|---|---|---|---|
| Huanglong dripping pills 2 | 0.164 | 0.195 | 0.215 | 0.226 | 0.231 | 0.249 | 0.255 |
| Huanglong dripping pills 1 | 0.092 | 0.126 | 0.147 | 0.159 | 0.168 | 0.179 | 0.191 |

TABLE 2
Moisture experiment results of Huanglong dripping pills at 25° C. (0 to 28 days)

| 25° C. moisture | day 0 | day 2 | day 4 | day 7 | day 10 | day 14 | day 28 |
|---|---|---|---|---|---|---|---|
| Huanglong dripping pills 2 | 1.72% | 2.11% | 2.29% | 2.33% | 2.48% | 2.58% | 2.66% |
| Huanglong dripping pills 1 | 0.91% | 1.31% | 1.58% | 1.71% | 1.86% | 1.92% | 2.09% |

As can be seen by comparing Table 1 and Table 2, the water activity of the Huanglong dripping pills 1 obtained in Example 1 is lower than that of the Huanglong dripping pills 2 obtained in Comparative example 1, indicating good tolerability of the Huanglong dripping pills 1 obtained in Example 1.

### Test Example 2

### Microbiological Experiments

Buffer: 0.9% NaCl solution containing 0.05% tween-80 (sterilized at 121° C. for 15 min).
Preparation of Huanglong dripping pill solution: 0.5 g of Huanglong dripping pills was added to 49.5 g of sterilized buffer, and homogenization was performed at 18000 rpm for 80 s.
Culture medium: (1) Plate count agar (PCA) (HB0101, QINGDAO HI-TECH INDUSTRIAL PARK HOPE BIO-TECHNOLOGY CO., LTD.); (2) Wilkins-Chalgren agar (WCA) (HB0261, QINGDAO HI-TECH INDUSTRIAL PARK HOPE BIO-TECHNOLOGY CO., LTD.); (3) MRS medium (027315, GUANGDONG HUANKAI MICROBIAL SCI. & TECH. CO., LTD.), with supplementary reagents (SR0370 mupirocin lithium salt and cysteine hydrochloride) added per manufacturer's instructions.
Culture method: The prepared Huanglong dripping pill solution was serially diluted with the buffer to appropriate concentrations, and then plated. PCA was used to culture total aerobic bacteria, and culture was performed at 37° C. after plating; WCA was used to culture total anaerobic bacteria, and anaerobic culture was performed at 37° C. after plating; and MRA is used to culture Bifidobacterium, and anaerobic culture was performed at 37° C. after plating. After 48 h, plate counting was performed. The results are as shown in Table 3 and Table 4.

Where Bifidobacterium was cultured with reference to Chinese National Standard GB4789.34-2016, using MRS medium+mupirocin lithium salt and cysteine hydrochloride.

TABLE 3
Microbiological experiment results of Huanglong dripping pills 2 at 25° C. (0 to 28 days)
Huanglong dripping pills 2

| | Total aerobic | | Total | | | |
|---|---|---|---|---|---|---|
| | bacteria | Survival | anaerobic | Survival | Bifidobacterium | Survival |
| Time | cfu/g | rate % | bacteria cfu/g | rate % | cfu/g | rate % |
| day 0 | 4.48E+06 | 100 | 4.92E+07 | 100 | 2.01E+07 | 100 |
| | 4.51E+06 | | 4.85E+07 | | 1.96E+07 | |
| | 4.60E+06 | | 4.87E+07 | | 1.94E+07 | |
| day 2 | 4.41E+06 | 98.3 | 4.69E+07 | 98.2 | 1.89E+07 | 95.3 |
| | 4.42E+06 | | 4.81E+07 | | 1.92E+07 | |
| | 4.52E+06 | | 4.87E+07 | | 1.80E+07 | |
| day 4 | 4.01E+06 | 87.4 | 4.36E+07 | 86.1 | 1.38E+07 | 68.2 |
| | 3.92E+06 | | 4.19E+07 | | 1.29E+07 | |
| | 3.92E+06 | | 4.05E+07 | | 1.35E+07 | |
| day 7 | 3.22E+06 | 70.2 | 2.08E+07 | 40.2 | 1.03E+07 | 54.4 |
| | 3.14E+06 | | 1.92E+07 | | 1.10E+07 | |
| | 3.18E+06 | | 1.88E+07 | | 1.08E+07 | |
| day 10 | 2.92E+06 | 62.3 | 1.52E+07 | 29.6 | 5.89E+06 | 29.9 |
| | 2.79E+06 | | 1.46E+07 | | 5.93E+06 | |
| | 2.75E+06 | | 1.34E+07 | | 5.85E+06 | |
| day 14 | 2.21E+06 | 50.1 | 1.21E+07 | 28.2 | 5.09E+06 | 25.7 |
| | 2.19E+06 | | 1.39E+07 | | 5.01E+06 | |
| | 2.38E+06 | | 1.51E+07 | | 5.08E+06 | |
| day 28 | 2.01E+06 | 42.2 | 1.08 E+07 | 19.9 | 4.01E+06 | 20.3 |
| | 1.92E+06 | | 9.88E+06 | | 3.92E+06 | |
| | 1.80E+06 | | 9.95E+06 | | 4.04E+06 | |

TABLE 4
Microbiological experiment results of Huanglong dripping pills 1 at 25° C. (0 to 28 days)
Huanglong dripping pills 1

| | Total aerobic | | Total | | | |
|---|---|---|---|---|---|---|
| | bacteria | Survival | anaerobic | Survival | Bifidobacterium | Survival |
| | cfu/g | rate % | bacteria cfu/g | rate % | cfu/g | rate % |
| day 0 | 4.86E+06 | 100 | 5.34E+07 | 100 | 2.17E+07 | 100 |
| | 4.91E+06 | | 5.29E+07 | | 2.09E+07 | |
| | 4.90E+06 | | 5.30E+07 | | 2.19E+07 | |
| day 2 | 4.88E+06 | 99.3 | 5.29E+07 | 99.6 | 2.05E+07 | 98.9 |
| | 4.76E+06 | | 5.30E+07 | | 2.12E+07 | |
| | 4.91E+06 | | 5.25E+07 | | 2.19E+07 | |
| day 4 | 4.28E+06 | 89.6 | 4.79E+07 | 88.4 | 1.77E+07 | 81.3 |
| | 4.36E+06 | | 4.70E+07 | | 1.82E+07 | |
| | 4.50E+06 | | 4.58E+07 | | 1.63E+07 | |
| day 7 | 3.91E+06 | 78.6 | 2.79E+07 | 50.3 | 1.42E+07 | 62.3 |
| | 3.88E+06 | | 2.60E+07 | | 1.29E+07 | |
| | 3.73E+06 | | 2.62E+07 | | 1.28E+07 | |
| day 10 | 3.21E+06 | 64.3 | 2.19E+07 | 41.2 | 9.52E+06 | 44.2 |
| | 3.09E+06 | | 2.10E+07 | | 9.58E+06 | |
| | 3.12E+06 | | 2.25E+07 | | 9.40E+06 | |
| day 14 | 2.99E+06 | 58.7 | 2.01E+07 | 36.8 | 7.83E+06 | 36.3 |
| | 2.79E+06 | | 1.98E+07 | | 7.88E+06 | |
| | 2.83E+06 | | 1.86E+07 | | 7.69E+06 | |
| day 28 | 2.49E+06 | 49.8 | 1.59E+07 | 29.4 | 6.62E+06 | 29.9 |
| | 2.39E+06 | | 1.48E+07 | | 6.39E+06 | |
| | 2.41E+06 | | 1.61E+07 | | 6.25E+06 | |

As can be seen by comparing Table 3 and Table 4, the stability of the Huanglong dripping pills 1 obtained by the drying method provided by the present application is superior that of the Huanglong dripping pills 2 obtained in Comparative example 1.

### Test Example 3

### In Vitro Simulated Gastrointestinal Tolerability Test

The dissolution test was performed according to Method III (small vessel method) of “0931 Dissolution and Release Test” in Chinese Pharmacopoeia 2020 Edition, Volume IV, using 200 mL of dissolution medium at a rotating speed of 50 rpm. After 2 h in a simulated gastric fluid, the dripping pills were transferred to a simulated intestinal fluid for 4 h before sampling. The survival rate of microorganisms in the test sample after dissolution treatment was determined using the “Viable Count Method for Probiotics” (with the survival rate of microorganisms at 0 h without dissolution treatment set as 100% for calculating the relative survival rate, expressed as the mean value of six dissolution vessels).

The method for preparing the simulated gastric fluid (dissolution medium) included: taking 3.84 mL of hydrochloric acid, adding about 800 mL of water and 10 g of pepsin, shaking well the resulting mixture, and diluting the resulting mixture to 1000 mL by adding water to obtain the simulated gastric fluid.

The method for preparing the simulated intestinal fluid (dissolution medium) included: taking 6.8 g of potassium dihydrogen phosphate, adding 500 mL for dissolving, and adjusting the pH value to 6.8 with 0.1 mol/L sodium hydroxide solution; taking 10 g of pancreatin and 3 g of bile salt, adding an appropriate amount of water for dissolving, mixing the two solutions, and diluting the resulting mixture to 1000 mL by adding water to obtain the simulated intestinal fluid.

TABLE 5
In vitro simulated gastrointestinal tolerability test of Huanglong dripping pills

| | | | | | Survival |
|---|---|---|---|---|---|
| Viable count (CFU/g) | 1# | 2# | 3# | Mean value | rate % |

Total anaerobic viable count

| | | | | | |
|---|---|---|---|---|---|
| Total anaerobic viable count of | 4.92E+07 | 4.85E+07 | 4.87E+07 | 4.88E+07 | 100 |
| Huanglong dripping pills 2 | | | | | |
| Gastric fluid (pH 1.2) 2 h | 3.92E+07 | 3.82E+07 | 3.84E+07 | 3.86E+07 | 79.1 |
| Gastric fluid (pH 1.2) 2 h + | 2.02E+07 | 1.89E+07 | 1.82E+07 | 1.91E+07 | 39.2 |
| intestinal fluid 4 h | | | | | |
| Total anaerobic viable count of | 5.34E+07 | 5.29E+07 | 5.30E+07 | 5.31E+07 | 100 |
| Huanglong dripping pills 1 | | | | | |
| Gastric fluid (pH 1.2) 2 h | 4.48E+07 | 4.32E+07 | 4.34E+07 | 4.38E+07 | 82.6 |
| Gastric fluid (pH 1.2) 2 h + | 2.22E+07 | 2.14E+07 | 2.18E+07 | 2.18E+07 | 41.1 |
| intestinal fluid 4 h | | | | | |
| Total anaerobic viable count of | 5.03E+08 | 5.04E+08 | 4.96E+08 | 5.01E+08 | 100 |
| microbiota powder | | | | | |
| Gastric fluid (pH 1.2) 2 h | 1.50E+06 | 1.50E+06 | 1.50E+06 | 1.50E+06 | 0.003 |
| Gastric fluid (pH 1.2) 2 h + | NA | NA | NA | NA | Not |
| intestinal fluid 4 h | | | | | detected |

Total anaerobic viable count

| | | | | | |
|---|---|---|---|---|---|
| Total aerobic viable count of | 4.48E+06 | 4.51E+06 | 4.60E+06 | 4.53E+06 | 100 |
| Huanglong dripping pills 2 | | | | | |
| Gastric fluid (pH 1.2) 2 h | 3.11E+06 | 2.91E+06 | 3.04E+06 | 3.02E+06 | 66.8 |
| Gastric fluid (pH 1.2) 2 h + | 1.79E+06 | 1.61E+06 | 1.67E+06 | 1.69E+06 | 37.4 |
| intestinal fluid 4 h | | | | | |
| Total aerobic viable count of | 4.86E+06 | 4.91E+06 | 4.90E+06 | 4.89E+06 | 100 |
| Huanglong dripping pills 1 | | | | | |
| Gastric fluid (pH 1.2) 2 h | 3.58E+06 | 3.41E+06 | 3.45E+06 | 3.48E+06 | 71.2 |
| Gastric fluid (pH 1.2) 2 h + | 2.05E+06 | 1.91E+06 | 1.89E+06 | 1.95E+06 | 39.9 |
| intestinal fluid 4 h | | | | | |
| Total aerobic viable count of | 6.34E+07 | 6.29E+07 | 6.36E+07 | 6.33E+07 | 100 |
| microbiota powder | | | | | |
| Gastric fluid (pH 1.2) 2 h | 5.06E+05 | 5.06E+05 | 5.06E+05 | 5.06E+05 | 0.008 |
| Gastric fluid (pH 1.2) 2 h + | NA | NA | NA | NA | Not |
| intestinal fluid 4 h | | | | | detected |

Bifidobacterium viable count

| | | | | | |
|---|---|---|---|---|---|
| Bifidobacterium viable count of | 2.01E+07 | 1.96E+07 | 1.94E+07 | 1.97E+07 | 100 |
| Huanglong dripping pills 2 | | | | | |
| Gastric fluid (pH 1.2) 2 h | 1.66E+07 | 1.52E+07 | 1.62E+07 | 1.60E+07 | 81.6 |
| Gastric fluid (pH 1.2) 2 h + | 9.96E+06 | 9.77E+06 | 9.85E+06 | 9.86E+06 | 50.1 |
| intestinal fluid 4 h | | | | | |
| Bifidobacterium viable count of | 2.17E+07 | 2.09E+07 | 2.19E+07 | 2.15E+07 | 100 |
| Huanglong dripping pills 1 | | | | | |
| Gastric fluid (pH 1.2) 2 h | 1.95E+07 | 1.81E+07 | 1.82E+07 | 1.86E+07 | 86.7 |
| Gastric fluid (pH 1.2) 2 h + | 1.22E+07 | 1.11E+07 | 1.06E+07 | 1.13E+07 | 52.6 |
| intestinal fluid 4 h | | | | | |
| Bifidobacterium viable count of | 2.50E+08 | 2.54E+08 | 2.49E+08 | 2.51E+08 | 100 |
| microbiota powder | | | | | |
| Gastric fluid (pH 1.2) 2 h | 1.12E+06 | 1.12E+06 | 1.12E+06 | 1.12E+06 | 0.0045 |
| Gastric fluid (pH 1.2) 2 h + | NA | NA | NA | NA | Not |
| intestinal fluid 4 h | | | | | detected |

For the Huanglong dripping pills 2, after treatment with pH 1.2 gastric acid solution plus enzyme for 2 h, the survival rate of total anaerobic bacteria was 79.1%, and after further treatment with pH 6.8 phosphate plus pancreatin buffer for 4 h, the survival rate was 39.2%.

For the Huanglong dripping pills 2, after treatment with pH 1.2 gastric acid solution plus enzyme for 2 h, the survival rate of total aerobic bacteria was 66.8%, and after further treatment with pH 6.8 phosphate plus pancreatin buffer for 4 h, the survival rate was 37.4%.

For the Huanglong dripping pills 2, after treatment with pH 1.2 gastric acid solution plus enzyme for 2 h, the survival rate of Bifidobacterium was 81.6%, and after further treatment with pH 6.8 phosphate plus pancreatin buffer for 4 h, the survival rate was 50.1%.

For the Huanglong dripping pills 1, after treatment with pH 1.2 gastric acid solution plus enzyme for 2 h, the survival rate of total anaerobic bacteria was 82.6%, and after further treatment with pH 6.8 phosphate plus pancreatin buffer for 4 h, the survival rate was 41.1%.

For the Huanglong dripping pills 1, after treatment with pH 1.2 gastric acid solution plus enzyme for 2 h, the survival rate of total aerobic bacteria was 71.2%, and after further treatment with pH 6.8 phosphate plus pancreatin buffer for 4 h, the survival rate was 39.9%.

For the Huanglong dripping pills 1, after treatment with pH 1.2 gastric acid solution plus enzyme for 2 h, the survival rate of Bifidobacterium was 86.7%, and after further treatment with pH 6.8 phosphate plus pancreatin buffer for 4 h, the survival rate was 52.6%.

The Huanglong dripping pills 1 obtained by the drying method provided by the present application exhibit better tolerability than the Huanglong dripping pills 2 obtained in Comparative example 1.

The foregoing descriptions are merely preferred embodiments of the present application, and it should be noted that for those of ordinary skill in the art, without departing from the principles of the present application, some improvements and refinement may also be made, which should also be considered as the protection scope of the present application.

What is claimed is:

1. A method for drying fecal microbiota multilayer dripping pills, wherein the method comprises the steps of:
providing fecal microbiota multilayer dripping pills;
performing centrifugal deoiling on the fecal microbiota multilayer dripping pills by using a deoiling agent, and then performing separation treatment to obtain deoiled dripping pills, wherein the deoiling agent is a mixture of fumed silica, soybean fiber, and oat cellulose;
performing blast drying on the deoiled dripping pills to obtain pre-dried Huanglong dripping pills; and
performing negative pressure drying on the pre-dried Huanglong dripping pills to obtain Huanglong dripping pills.

2. The drying method of claim 1, wherein a mass ratio of the fumed silica to the soybean fiber to the oat cellulose is 1:1 to 2:1 to 2.

3. The drying method of claim 1 further comprising a method for preparing the deoiling agent that comprises the steps of:
mixing fumed silica, soybean fiber, and oat cellulose, and
performing vacuum drying and cooling to obtain the deoiling agent, wherein in the vacuum drying, a temperature is 40° C. to 60° C., and a time is 4 to 6 h.

4. The drying method of claim 1, wherein the mass ratio of the fecal microbiota multilayer dripping pills to the deoiling agent is 1:1 to 3.

5. The drying method of claim 1, wherein in the centrifugal deoiling, a rotating speed is 500 to 1200 rpm, and a time is 10 to 15 min.

6. The drying method of claim 1, wherein the centrifugal deoiling comprises: loading the deoiling agent and the fecal microbiota multilayer dripping pills into a sterile bag, and then placing the sterile bag in a centrifuge for centrifugal deoiling.

7. The drying method of claim 1, wherein a time for the separation treatment is 3 to 8 min;
the separation treatment is performed in a rotary drying drum, the drum wall of the rotary drying drum is provided with pores having a pore size of 0.5 to 1.5 mm, and a rotating speed of the rotary drying drum is 5 to 10 rpm.

8. The drying method of claim 1, wherein a rotating speed of the blast drying is 4 to 10 rpm, and a time of the blast drying is 5.5 to 7.5 h; a blast gas of the blast drying is sterile air;
the blast drying is performed in a rotary drying drum, the drum wall of the rotary drying drum is provided with pores having a pore size of 0.5 to 1.5 mm, and the rotary drying drum is lined with a pure cotton oil-absorbing cloth;
during the first 60 min of the blast drying, the pure cotton oil-absorbing cloth is replaced every 30 min.

9. The drying method of claim 1, wherein the pre-dried Huanglong dripping pills have a water activity of 0.15 to 0.2.

10. The drying method of claim 1, wherein in the negative pressure drying, the pressure is 0 to 0.02 MPa, the temperature is 25° C. to 28° C., the humidity is 3 to 15%, and the time is 60 to 180 min.

11. The drying method of claim 1, wherein the negative pressure drying is performed in a rotary drying drum, and the rotating speed of the rotary drying drum is 4 to 10 rpm;
the rotary drying drum is filled with a desiccant, the desiccant is a mixture of a color changing silica gel and anhydrous calcium chloride, and the mass ratio of the color changing silica gel to the anhydrous calcium chloride is 1:1 to 3.

12. The drying method of claim 11, wherein the color changing silica gel and the anhydrous calcium chloride have particle sizes of 5 to 8 mm, independently.

13. The drying method of claim 11, further comprising a method for preparing the desiccant that comprises the steps of:
mixing a color changing silica gel and anhydrous calcium chloride, and
performing drying to obtain the desiccant, wherein in the drying, the temperature is 150° C. to 170° C., and the time is 6 to 8 h.

14. The drying method of claim 1, wherein the Huanglong dripping pills have a water activity of 0.08 to 0.14.

15. The drying method of claim 3, wherein the method for preparing the deoiling agent comprises the steps of:
mixing fumed silica, soybean fiber, and oat cellulose, and
performing vacuum drying and cooling to obtain the deoiling agent, wherein in the vacuum drying, a temperature is 40° C. to 60° C., and a time is 4 to 6 h.

16. The drying method of claim 2, wherein the mass ratio of the fecal microbiota multilayer dripping pills to the deoiling agent is 1:1 to 3.

17. The drying method of claim 5, wherein the centrifugal deoiling comprises:
loading the deoiling agent and the fecal microbiota multilayer dripping pills into a sterile bag, and then placing the sterile bag in a centrifuge for centrifugal deoiling.

18. The drying method of claim 8, wherein the pre-dried Huanglong dripping pills have a water activity of 0.15 to 0.2.

19. The drying method of claim 10, wherein the negative pressure drying is performed in a rotary drying drum, and the rotating speed of the rotary drying drum is 4 to 10 rpm;
the rotary drying drum is filled with a desiccant, the desiccant is a mixture of a color changing silica gel and anhydrous calcium chloride, and the mass ratio of the color changing silica gel to the anhydrous calcium chloride is 1:1 to 3.
